SEQUENCE LISTING (2 sequences)

SEQ ID NO: 1 - DNA, 1411 bp, Bifidobacterium animalis subsp. lactis
acggctcccccacaagggtcgggccaccggcttcgggtgctacccactttcatgacttgacgggcggtgtgtacaaggcccgggaacgcattcaccgcggcgttgctgatccgcgattactagcgactccgccttcacgcagtcgagttgcagactgcgatccgaactgagaccggttttcagcgatccgccccacgtcaccgtgtcgcaccgcgttgtaccggccattgtagcatgcgtgaagccctggacgtaaggggcatgatgatctgacgtcatccccaccttcctccgagttgaccccggcggtcccacatgagttcccggcatcacccgctggcaacatgcggcgagggttgcgctcgttgcgggacttaacccaacatctcacgacacgagctgacgacgaccatgcaccacctgtgaaccggccccgaagggaaaccgtgtctccacggcgatccggcacatgtcaagcccaggtaaggttcttcgcgttgcatcgaattaatccgcatgctccgccgcttgtgcgggcccccgtcaatttctttgagttttagccttgcggccgtactccccaggcgggatgcttaacgcgttggctccgacacgggacccgtggaaagggccccacatccagcatccaccgtttacggcgtggactaccagggtatctaatcctgttcgctccccacgctttcgctcctcagcgtcagtgacggcccagagacctgccttcgccattggtgttcttcccgatatctacacattccaccgttacaccgggaattccagtctcccctaccgcactccagcccgcccgtacccggcgcagatccaccgttaggcgatggactttcacaccggacgcgacgaaccgcctacgagccctttacgcccaataaatccggataacgctcgcaccctacgtattaccgcggctgctggcacgtagttagccggtgcttattcgaacaatccactcaacacggccgaaaccgtgccttgcccttgaacaaaagcggtttacaacccgaaggcctccatcccgcacgcggcgtcgctgcatcaggcttgcgcccattgtgcaatattccccactgctgcctcccgtaggagtctgggccgtatctcagtcccaatgtggccggtcaccctctcaggccggctacccgtcaacgccttggtgggccatcaccccgccaacaagctgataggacgcgaccccatcccatgccgcaaaagcatttcccaccccaccatgcgatggagcggagcatccggtattaccacccgtttccaggagctattccggtgcacagggcaggttggtcacgcattactcacccgttcgccactctcacccgacagcaagctgccagggatcccgttcgactgcatgtgtaag

SEQ ID NO: 2 - DNA, 994 bp, Bifidobacterium animalis subsp. lactis
ggatctcgatgagagcagcgtggtatcaccatcaacattgcccacatcgagtaccagacggccaagcgtcactacgcccacgtcgactgcccgggccacgccgacttcgtgaagaacatgatcaccggcgctgcccagatggatggcgccatcctcgttgtggccgccaccgacggcccgatggcccagacccgcgagcacgtgctgctcgcccgtcaggtcggcgtcccgaagatcctcgtcgctctgaacaagtgcgatatggtcgatgacgaagagctcatcgagctcgtcgaagaagaggtccgcgacctcctcgacgagaacggcttcgaccgcgactgcccggtcgtgcacacctccgcttacggcgctctgcatgacgacgctcccggatcacgacaagtgggttgccaccatcaaggagctcatggacgacgtcgacgagtacatcccgaccccggtccacgacctcgacaagccgttcctgatgccgatcgaggacgtcttcaccatctccggccgtggcaccgtcgtcaccggtcgtgtcgagcgcggcaagctgccgatcaacacgaacgtcgagatcgtcggcatccgcccgacccagaccaccaccgtcacctccatcgagaccttccacaagcagatggatgagtgcgaggccggcgacaacaccggtctgctgctccgcggcatcaaccgcaccgacgtcgagcgtggccaggtcgtggctgctccgggttcggtcaccccgcacaccaagttcgaaggcgaagtctacgtccttaccaaggatgagggcggccgtcactcgccgttcttctcgaactaccgtccgcagttctacttccgcaccaccgacgtcaccggcgtcatcacgctgccggaaggcgtcgagatggttcagcctggcgatcacgcgaccttcacggttgagctgatccagccgatcgctatggaagagggcttcaccttcccagtgcttgaaggc

(19) United States
(12) Patent Application Publication
(10) Pub. No.: US 2023/0034193 A1
(43) Pub. Date: Feb. 2, 2023

(54) BIFIDOBACTERIUM ANIMALIS SUBSP. LACTIS I797, METHOD FOR SEPARATION AND PURIFICATION THEREOF, AND USE THEREOF

(71) Applicant: JUNLEBAO DAIRY GROUP CO., LTD., Hebei (CN)

(72) Inventors: Lihua WEI, Shijiazhuang (CN); Lili FENG, Shijiazhuang (CN); Dong ZHANG, Shijiazhuang (CN); Hong ZHU, Shijiazhuang (CN); Shijie WANG, Shijiazhuang (CN); Yiping XUN, Shijiazhuang (CN); Yuling XUE, Shijiazhuang (CN); Zhiyuan KANG, Shijiazhuang (CN); Xiaowei HAN, Shijiazhuang (CN)

(21) Appl. No.: 17/784,654

(22) PCT Filed: Jul. 13, 2020
(86) PCT No.: PCT/CN2020/101661

§ 371 Date: Jun. 12, 2022

(30) Foreign Application Priority Data
Dec. 11, 2019 (CN) 201911263293.1
Apr. 3, 2020 (CN) 202010259210.8

(51) Int. Cl. A61K 35/745; C12N 1/20; A23L 33/135; A23L 2/38; A23C 9/123; A23L 7/104; A23L 13/40

(52) U.S. Cl. CPC A61K 35/745; C12N 1/205; A23L 33/135; A23L 2/382; A23C 9/1234; A23L 7/104; A23L 13/45; C12R 2001/01

(57) ABSTRACT

Provided are a Bifidobacterium animalis subsp. lactis i797, a method for the separation and purification thereof, and a use thereof. The strain was is deposited in the China General Microbiological Culture Collection Center on Aug. 20, 2019, wherein the deposit address is Building 3, No. 1 Beichen West Road, Chaoyang District, Beijing, and the deposit number is CGMCC NO. 18403. The Bifidobacterium animalis subsp. lactis i797 can adjust the balance of intestinal flora, improve stool characteristics, and has a better survival rate in simulated digestive juice; in addition, after being stored at 37° C., a relatively high temperature which is suitable for the growth of lactic acid bacteria, same can successfully control post-acidification.

### I. TECHNICAL FIELD

The present invention belongs to the technical field of bioengineering, and relates to a strain, a screening method and use thereof, in particular to Bifidobacterium animalis subsp. lactis i797, method for separation and purification thereof and use thereof.

### II. BACKGROUND ART

Bifidobacterium was first discovered when Henry Tissier separated it from the feces of breast-fed babies in 1899. Bifidobacterium belongs to Gram-positive bacilli, is very sensitive to oxygen, has poor tolerance to low PH and is easily inactivated in a low PH environment; the optimum PH for Bifidobacterium is 6.5-7.0, and the optimum growth temperature for Bifidobacterium is 37-42° C. Bifidobacterium widely exists in habitats such as digestive tracts, vaginas and oral cavities of human and animal, and is one of the important members of the intestinal flora of human and animal. It has been found that there are 32 subtypes of Bifidobacterium, and there are up to 70 kinds of biological agents containing Bifidobacterium.

Bifidobacterium is a kind of probiotic bacteria that neither produces endotoxins or exotoxins, nor produces pathogenic substances or harmful gases, which is of great significance for maintaining the health of the body. Bifidobacterium can effectively maintain the normal balance of the intestinal flora, and has unique physiological functions against diarrhea, constipation, infection and tumor etc. Bifidobacterium plays an important role in body health, for example, maintaining intestinal microecological balance, inhibiting invasion and colonization of pathogenic bacteria, regulating body immunity and reducing cholesterol content, etc. Therefore, it has a wide application prospect. In addition, the nutrient substances such as vitamins and amino acids produced by Bifidobacterium during the growing process can improve the nutritional value of milk. Therefore, Bifidobacterium is widely applied in milk production.

Bifidobacterium can colonize in the intestinal tract of a newborn baby within a few hours after the birth. The quantity of Bifidobacterium distributed in the gastrointestinal tract decreases as the age increases, and is highest in breast-fed babies. In infants, Bifidobacterium accounts for about 60% of the total intestinal bacteria; in old people over sixty, Bifidobacterium accounts for only 7.9% of the total intestinal bacteria, while putrefactive bacteria such as Clostridium perfringens and Escherichia coli increase greatly; in elderly people, the intestinal tracts are full of putrefactive bacteria, while Bifidobacterium almost disappears. The researches demonstrate that the minimum viable bacteria concentration should be higher than 107 cfu/mL to maintain the function of probiotic bacteria.

Therefore, maintaining the quantity of Bifidobacterium in the intestinal tracts is very important to maintain human health. Research on the mechanism of Bifidobacterium for regulating the intestinal flora is helpful for the industrial application of Bifidobacterium. For example, up to now, some Bifidobacterium strains have been put into industrial use in China to treat the diseases caused by intestinal flora disorder, such as diarrhea and functional constipation, etc., and have achieved remarkable results. Besides, some medical studies demonstrate that long-term use of artificially synthesized probiotic bacteria may result in the phenomenon of intestinal tract gradually losing its ability to reproduce probiotic bacteria by itself and dependency of intestinal tract on artificial probiotic bacteria arising, leading to “probiotic dependence”.

In recent years, the researches on the mechanism of Bifidobacterium for regulating the intestinal flora mainly focused on colonization, specific binding and generated metabolites of Bifidobacterium. However, in-depth researches on how to give full play to the function of Bifidobacterium for regulating the intestinal flora should be carried out, and such researches involve selection and cultivation of strains.

### III. CONTENTS OF THE INVENTION

An object of the present invention is to provide a Bifidobacterium animalis subsp. lactis i797, which is separated and screened from the feces of breast-fed infants or babies, and can regulate the balance of the intestinal flora and promote body health.

Another object of the present invention is to provide a method for separating and purifying the above Bifidobacterium animalis subsp. lactis i797.

Another object of the present invention is to provide use of the above Bifidobacterium animalis subsp. lactis i797.

To attain the objects described above, the present invention employs the following technical solution:

A Bifidobacterium animalis subsp. lactis i797, preserved in the China General Microbiological Culture Collection Center (address: No. 3, Yard No. 1, Beichen West Road, Chaoyang District, Beijing) on Aug. 20, 2019, with a preservation number of CGMCC No. 18403.

As a limitation, the Bifidobacterium animalis subsp. lactis i797 is screened out from the intestinal flora of infants or babies.

As a second limitation, its 16SrRNA sequence is as follows:

ACGGCTCCCCCACAAGGGTCGGGCCACCGGCTTCGGGTGCTACCCACTTT
CATGACTTGACGGGCGGTGTGTACAAGGCCCGGGAACGCATTCACCGCGG
CGTTGCTGATCCGCGATTACTAGCGACTCCGCCTTCACGCAGTCGAGTTG
CAGACTGCGATCCGAACTGAGACCGGTTTTCAGCGATCCGCCCCACGTCA
CCGTGTCGCACCGCGTTGTACCGGCCATTGTAGCATGCGTGAAGCCCTGG
ACGTAAGGGGCATGATGATCTGACGTCATCCCCACCTTCCTCCGAGTTGA
CCCCGGCGGTCCCACATGAGTTCCCGGCATCACCCGCTGGCAACATGCGG
CGAGGGTTGCGCTCGTTGCGGGACTTAACCCAACATCTCACGACACGAGC
TGACGACGACCATGCACCACCTGTGAACCGGCCCCGAAGGGAAACCGTGT
CTCCACGGCGATCCGGCACATGTCAAGCCCAGGTAAGGTTCTTCGCGTTG
CATCGAATTAATCCGCATGCTCCGCCGCTTGTGCGGGCCCCCGTCAATTT
CTTTGAGTTTTAGCCTTGCGGCCGTACTCCCCAGGCGGGATGCTTAACGC
GTTGGCTCCGACACGGGACCCGTGGAAAGGGCCCCACATCCAGCATCCAC
CGTTTACGGCGTGGACTACCAGGGTATCTAATCCTGTTCGCTCCCCACGC
TTTCGCTCCTCAGCGTCAGTGACGGCCCAGAGACCTGCCTTCGCCATTGG
TGTTCTTCCCGATATCTACACATTCCACCGTTACACCGGGAATTCCAGTC
TCCCCTACCGCACTCCAGCCCGCCCGTACCCGGCGCAGATCCACCGTTAG
GCGATGGACTTTCACACCGGACGCGACGAACCGCCTACGAGCCCTTTACG
CCCAATAAATCCGGATAACGCTCGCACCCTACGTATTACCGCGGCTGCTG
GCACGTAGTTAGCCGGTGCTTATTCGAACAATCCACTCAACACGGCCGAA
ACCGTGCCTTGCCCTTGAACAAAAGCGGTTTACAACCCGAAGGCCTCCAT
CCCGCACGCGGCGTCGCTGCATCAGGCTTGCGCCCATTGTGCAATATTCC
CCACTGCTGCCTCCCGTAGGAGTCTGGGCCGTATCTCAGTCCCAATGTGG
CCGGTCACCCTCTCAGGCCGGCTACCCGTCAACGCCTTGGTGGGCCATCA
CCCCGCCAACAAGCTGATAGGACGCGACCCCATCCCATGCCGCAAAAGCA
TTTCCCACCCCACCATGCGATGGAGCGGAGCATCCGGTATTACCACCCGT
TTCCAGGAGCTATTCCGGTGCACAGGGCAGGTTGGTCACGCATTACTCAC
CCGTTCGCCACTCTCACCCGACAGCAAGCTGCCAGGGATCCCGTTCGACT
GCATGTGTAAG.

As a third limitation, its tuf gene sequence is as follows:

GGATCTCGATGAGAGCAGCGTGGTATCACCATCAACATTGCCCACATCGA
GTACCAGACGGCCAAGCGTCACTACGCCCACGTCGACTGCCCGGGCCACG
CCGACTTCGTGAAGAACATGATCACCGGCGCTGCCCAGATGGATGGCGCC
ATCCTCGTTGTGGCCGCCACCGACGGCCCGATGGCCCAGACCCGCGAGCA
CGTGCTGCTCGCCCGTCAGGTCGGCGTCCCGAAGATCCTCGTCGCTCTGA
ACAAGTGCGATATGGTCGATGACGAAGAGCTCATCGAGCTCGTCGAAGAA
GAGGTCCGCGACCTCCTCGACGAGAACGGCTTCGACCGCGACTGCCCGGT
CGTGCACACCTCCGCTTACGGCGCTCTGCATGACGACGCTCCCGGATCAC
GACAAGTGGGTTGCCACCATCAAGGAGCTCATGGACGACGTCGACGAGTA
CATCCCGACCCCGGTCCACGACCTCGACAAGCCGTTCCTGATGCCGATCG
AGGACGTCTTCACCATCTCCGGCCGTGGCACCGTCGTCACCGGTCGTGTC
GAGCGCGGCAAGCTGCCGATCAACACGAACGTCGAGATCGTCGGCATCCG
CCCGACCCAGACCACCACCGTCACCTCCATCGAGACCTTCCACAAGCAGA
TGGATGAGTGCGAGGCCGGCGACAACACCGGTCTGCTGCTCCGCGGCATC
AACCGCACCGACGTCGAGCGTGGCCAGGTCGTGGCTGCTCCGGGTTCGGT
CACCCCGCACACCAAGTTCGAAGGCGAAGTCTACGTCCTTACCAAGGATG
AGGGCGGCCGTCACTCGCCGTTCTTCTCGAACTACCGTCCGCAGTTCTAC
TTCCGCACCACCGACGTCACCGGCGTCATCACGCTGCCGGAAGGCGTCGA
GATGGTTCAGCCTGGCGATCACGCGACCTTCACGGTTGAGCTGATCCAGC
CGATCGCTATGGAAGAGGGCTTCACCTTCCCAGTGCTTGAAGGC.

The present invention further provides a method for separating and purifying above Bifidobacterium animalis subsp. lactis i797, comprising the following steps which are carried out in sequence:

### I. Sample Collection

obtaining intestinal feces of infants or babies, then adding the intestinal feces into normal saline and mixing thoroughly to obtain a sample A;

### II. Sample Enrichment

adding the sample A into a modified MRS liquid culture medium, and culturing in an anaerobic environment at 35-40° C. for 62-82 hours to obtain a culture solution B;
wherein the modified MRS liquid culture medium is an MRS liquid culture medium added with 0.5 wt % cysteine;
the volume ratio of the sample A to the modified MRS liquid culture medium is 1:10-100;

### III. Strain Separation and Screening

diluting the culture solution B with 0.9% sterile normal saline by ten-time gradient multiplication, i.e., diluting to 10⁻¹, 10⁻², 10⁻³, 10⁻⁴ and 10⁻⁵ times sequentially, thus obtaining bacterial suspensions C₁-C₅ correspondingly;
melting the modified MRS solid culture medium and pouring it into first to fifth culture dishes, thus obtaining culture media D₁-D₅ after cooling and complete solidification; drawing 0.1 mL bacterial suspensions C₁-C₅ respectively, and spreading on the culture media D₁-D₅ respectively in one-to-one correspondence, then turning the plates upside down and culturing in an anaerobic environment at 35-40° C. for culture for 62-82 hours, and observing the growth of the colonies;
the modified MRS solid culture medium is a solid culture medium obtained by adding 15 wt % agar per 1,000 mL modified MRS liquid culture medium;
after typical colonies appear on the plates, picking corresponding single colony E;

### IV. Strain Purification

picking a selected single colony E and streak-inoculating the single colony E culture on the modified MRS solid culture medium, and culturing in anaerobic environment at 35-40° C. for 62-82 hours to obtain a single colony F;
streak-inoculating the single colony F further on the modified MRS solid culture medium, and culturing in an anaerobic environment at 35-40° C. for 62-82 hours to obtain a single colony G;
further streak-inoculating the single colony G on the modified MRS solid culture medium, and culturing in an anaerobic environment at 35-40° C. for 62-82 hours to obtain a pure culture H, which is the strain of Bifidobacterium animalis subsp. lactis i797.

As a limitation, the strain of Bifidobacterium animalis subsp. lactis i797 is preserved as follows: mixing the pure culture H with 50 wt % sterile glycerol at a ratio of 1:1, placing the mixture in a strain preservation tube, mixing homogeneously and then preserving at −80-70° C.; at the same time, inoculating on the test-tube slant of the modified MRS solid culture medium for temporary storage.

As a second limitation, the components of the modified MRS liquid culture medium include: casein peptone, beef extract, yeast extract, glucose, sodium acetate, diamine citrate, Tween-80, K₂HPO₄, MgSO₄.7H₂O, MnSO₄.7H₂O, cysteine and distilled water;

wherein a dosage ratio of casein peptone:beef extract:yeast extract:glucose:sodium acetate:diamine citrate:Tween-80:K₂HPO₄:MgSO₄.7H₂O:MnSO₄.7H₂O:cysteine:distilled water is 10 g:10 g:5 g:20 g:5 g:2 g:1 g:2 g:0.2 g:0.05 g:0.5 g:1,000 mL.

The present invention further provides use of the Bifidobacterium animalis subsp. lactis i797 in preparation of drinks, foods or medicines.

As a limitation, the drinks are beverages or fermented milk drinks;

the foods are cereals, cereal derivatives, fermented meat products, probiotics or milk foods;
the medicines are in dosage form of capsule, tablet, pill or powder.

As a further limitation, the probiotics are compound probiotics.

With the technical solution described above, compared with the prior art, the present invention achieves the following technical progresses:
(1) The Bifidobacterium animalis subsp. lactis i797 provided by the present invention has an outstanding intestinal regulation function, and can regulate the balance of the intestinal flora and improve the characteristics of feces;
(2) The experiments demonstrate: compared with Bifidobacterium BB-12, the Bifidobacterium animalis subsp. lactis i797 provided by the present invention has a better survival rate in simulated digestive fluids, which is up to 7.4% and obviously superior to that of Bifidobacterium BB-12; thus, the strain can play a better probiotic role;
(3) The Bifidobacterium animalis subsp. lactis i797 provided by the present invention can control the post-acidification well under a storage condition suitable for the growth of lactic acid bacteria at a higher temperature of 37° C.;
(4) The Bifidobacterium animalis subsp. lactis i797 provided by the present invention has a wide range of application, and can be used not only for preparing fermented dairy products, but also for producing other probiotic products.

The Bifidobacterium animalis subsp. lactis i797 provided by the present invention is suitable for regulating intestinal tract, balancing the intestinal flora, and improving the characteristics of feces.

### IV. EMBODIMENTS

Hereunder some preferred examples of the present invention will be detailed. It should be understood that the preferred examples described here are only used to describe and explain the present invention, but not intended to limit the present invention.

### Example 1. A Bifidobacterium animalis Subsp. Lactis i797

In this example, a Bifidobacterium animalis subsp. lactis i797, which is separated and screened from the feces of breast-fed infants or babies, and preserved in the China General Microbiological Culture Collection Center (address: No. 3, Yard No. 1, Beichen West Road, Chaoyang District, Beijing) on Aug. 20, 2019, with a preservation number of CGMCC No. 18403, is provided.

The 16SrRNA sequence of above Bifidobacterium animalis subsp. lactis i797 is as follows:

ACGGCTCCCCCACAAGGGTCGGGCCACCGGCTTCGGGTGCTACCCACTTT
CATGACTTGACGGGCGGTGTGTACAAGGCCCGGGAACGCATTCACCGCGG
CGTTGCTGATCCGCGATTACTAGCGACTCCGCCTTCACGCAGTCGAGTTG
CAGACTGCGATCCGAACTGAGACCGGTTTTCAGCGATCCGCCCCACGTCA
CCGTGTCGCACCGCGTTGTACCGGCCATTGTAGCATGCGTGAAGCCCTGG
ACGTAAGGGGCATGATGATCTGACGTCATCCCCACCTTCCTCCGAGTTGA
CCCCGGCGGTCCCACATGAGTTCCCGGCATCACCCGCTGGCAACATGCGG
CGAGGGTTGCGCTCGTTGCGGGACTTAACCCAACATCTCACGACACGAGC
TGACGACGACCATGCACCACCTGTGAACCGGCCCCGAAGGGAAACCGTGT
CTCCACGGCGATCCGGCACATGTCAAGCCCAGGTAAGGTTCTTCGCGTTG
CATCGAATTAATCCGCATGCTCCGCCGCTTGTGCGGGCCCCCGTCAATTT
CTTTGAGTTTTAGCCTTGCGGCCGTACTCCCCAGGCGGGATGCTTAACGC
GTTGGCTCCGACACGGGACCCGTGGAAAGGGCCCCACATCCAGCATCCAC
CGTTTACGGCGTGGACTACCAGGGTATCTAATCCTGTTCGCTCCCCACGC
TTTCGCTCCTCAGCGTCAGTGACGGCCCAGAGACCTGCCTTCGCCATTGG
TGTTCTTCCCGATATCTACACATTCCACCGTTACACCGGGAATTCCAGTC
TCCCCTACCGCACTCCAGCCCGCCCGTACCCGGCGCAGATCCACCGTTAG
GCGATGGACTTTCACACCGGACGCGACGAACCGCCTACGAGCCCTTTACG
CCCAATAAATCCGGATAACGCTCGCACCCTACGTATTACCGCGGCTGCTG
GCACGTAGTTAGCCGGTGCTTATTCGAACAATCCACTCAACACGGCCGAA
ACCGTGCCTTGCCCTTGAACAAAAGCGGTTTACAACCCGAAGGCCTCCAT
CCCGCACGCGGCGTCGCTGCATCAGGCTTGCGCCCATTGTGCAATATTCC
CCACTGCTGCCTCCCGTAGGAGTCTGGGCCGTATCTCAGTCCCAATGTGG
CCGGTCACCCTCTCAGGCCGGCTACCCGTCAACGCCTTGGTGGGCCATCA
CCCCGCCAACAAGCTGATAGGACGCGACCCCATCCCATGCCGCAAAAGCA
TTTCCCACCCCACCATGCGATGGAGCGGAGCATCCGGTATTACCACCCGT
TTCCAGGAGCTATTCCGGTGCACAGGGCAGGTTGGTCACGCATTACTCAC
CCGTTCGCCACTCTCACCCGACAGCAAGCTGCCAGGGATCCCGTTCGACT
GCATGTGTAAG.

Its tuf gene sequence is as follows:

GGATCTCGATGAGAGCAGCGTGGTATCACCATCAACATTGCCCACATCGA
GTACCAGACGGCCAAGCGTCACTACGCCCACGTCGACTGCCCGGGCCACG
CCGACTTCGTGAAGAACATGATCACCGGCGCTGCCCAGATGGATGGCGCC
ATCCTCGTTGTGGCCGCCACCGACGGCCCGATGGCCCAGACCCGCGAGCA
CGTGCTGCTCGCCCGTCAGGTCGGCGTCCCGAAGATCCTCGTCGCTCTGA
ACAAGTGCGATATGGTCGATGACGAAGAGCTCATCGAGCTCGTCGAAGAA
GAGGTCCGCGACCTCCTCGACGAGAACGGCTTCGACCGCGACTGCCCGGT
CGTGCACACCTCCGCTTACGGCGCTCTGCATGACGACGCTCCCGGATCAC
GACAAGTGGGTTGCCACCATCAAGGAGCTCATGGACGACGTCGACGAGTA
CATCCCGACCCCGGTCCACGACCTCGACAAGCCGTTCCTGATGCCGATCG
AGGACGTCTTCACCATCTCCGGCCGTGGCACCGTCGTCACCGGTCGTGTC
GAGCGCGGCAAGCTGCCGATCAACACGAACGTCGAGATCGTCGGCATCCG
CCCGACCCAGACCACCACCGTCACCTCCATCGAGACCTTCCACAAGCAGA
TGGATGAGTGCGAGGCCGGCGACAACACCGGTCTGCTGCTCCGCGGCATC
AACCGCACCGACGTCGAGCGTGGCCAGGTCGTGGCTGCTCCGGGTTCGGT
CACCCCGCACACCAAGTTCGAAGGCGAAGTCTACGTCCTTACCAAGGATG
AGGGCGGCCGTCACTCGCCGTTCTTCTCGAACTACCGTCCGCAGTTCTAC
TTCCGCACCACCGACGTCACCGGCGTCATCACGCTGCCGGAAGGCGTCGA
GATGGTTCAGCCTGGCGATCACGCGACCTTCACGGTTGAGCTGATCCAGC
CGATCGCTATGGAAGAGGGCTTCACCTTCCCAGTGCTTGAAGGC.

### Example 2. A Method for Separating and Purifying Bifidobacterium animalis Subsp. Lactis i797

In this example, a method for separating and purifying the Bifidobacterium animalis subsp. lactis i797 described in the example 1 is provided, including the following steps which are carried out in sequence:

### I. Sample Collection

1 g intestinal feces of infants or babies is obtained and then added into 9 mL normal saline for thorough mixing to obtain a sample A;

### II. Sample Enrichment

2 mL (V₁) sample A is added into 100 mL (V₂) modified MRS liquid culture medium, and cultured in an anaerobic environment at 37° C. (T₁) for 72 hours (t₁) to obtain a culture solution B;

### III. Strain Separation and Screening

1 mL culture solution B₁ is diluted with 0.9% sterile normal saline by ten-time gradient multiplication, i.e., diluting to 10⁻¹, 10⁻², 10⁻³, 10⁻⁴ and 10⁻⁵ times sequentially, thus obtaining bacterial suspensions C1₁-C1₅ correspondingly;

A modified MRS solid culture medium is melted and poured into the first to fifth culture dishes, thus culture media D₁-D₅ are obtained after cooling and complete solidification; 0.1 mL bacterial suspensions C₁-C₅ are drawn and spread on the culture media D₁-D₅ respectively in one-to-one correspondence, then the plates are turned upside down and cultured in an anaerobic environment at 37° C. (T₂) for 72 hours (t₂), and the growth of the colonies is observed;

After typical colonies appear on the plates, corresponding single colony E is selected according to the colony characteristics of standard Bifidobacterium with reference to images in relevant literature;

### IV. Strain Purification

A selected single colony E is picked and its culture is streak-inoculated on the modified MRS solid culture medium, and cultured in anaerobic environment at 37° C. (T₃) for 72 hours (t₃) to obtain a single colony F;

The single colony F is further streak-inoculated on the modified MRS solid culture medium, and cultured in an anaerobic environment at 37° C. (T₄) for 72 hours (t₄) to obtain a single colony G;

The single colony G is further streak-inoculated on the modified MRS solid culture medium, and cultured in an anaerobic environment at 37° C. (T₅) for 72 hours (t₅) to obtain a pure culture H, which is the strain of Bifidobacterium animalis subsp. lactis i797;

### V. Preservation

The pure culture H is mixed with 50 wt % sterile glycerol at a ratio of 1:1, the mixture is placed in a strain preservation tube, mixed homogeneously and then preserved at −70° C. (T₆); at the same time, it is inoculated on the test-tube slant of the modified MRS solid culture medium for temporary storage.

In this example, the raw materials of the modified MRS liquid culture medium include: casein peptone, beef extract, yeast extract, glucose, sodium acetate, diamine citrate, Tween-80, K₂HPO₄, MgSO₄.7H₂O, MnSO₄.7H₂O, cysteine and distilled water; wherein a dosage ratio of casein peptone:beef extract:yeast extract:glucose:sodium acetate:diamine citrate:Tween-80:K₂HPO₄:MgSO₄-7H₂O:MnSO₄.7H₂O:cysteine:distilled water is 10 g:10 g:5 g:20 g:5 g:2 g:1 g:2 g:0.2 g:0.05 g:0.5 g:1,000 mL; the modified MRS solid culture medium is obtained by adding 15 wt % agar per 1,000 mL modified MRS liquid culture medium.

### Examples 3-6. A Method for Separating and Purifying the Bifidobacterium animalis Subsp. lactis i797

The examples 3-6 provide a separation and purification method of the example 1 respectively, which is essentially the same as that of the example 2, except that the technical parameters of the separation and purification process are different. The specific parameters are shown in Table 1:

TABLE 1
Separation and Purification Process and Parameters in Examples 3-6

| | | Example | | | |
|---|---|---|---|---|---|
| Step | Parameter | 3 | 4 | 5 | 6 |
| II | V₁ (ml) | 2 | 2 | 2 | 2 |
| | V₂ (ml) | 20 | 70 | 200 | 154 |
| | T₁ (° C.) | 35 | 36.4 | 40 | 38 |
| | t₁ (h) | 82 | 78 | 62 | 69 |
| III | T₂ (° C.) | 39 | 40 | 36 | 35 |
| | t₂ (h) | 69.5 | 82 | 62 | 75.4 |
| IV | T₃ (° C.) | 40 | 35 | 36.3 | 37.5 |
| | t₃ (h) | 70 | 82 | 63 | 62 |
| | T₄ (° C.) | 35 | 38.1 | 39.6 | 40 |
| | t₄ (h) | 62 | 80 | 69 | 82 |
| | T₅ (° C.) | 37.2 | 35 | 40 | 35.9 |
| | t₅ (h) | 76 | 62 | 82 | 81 |
| V | T₆ (° C.) | −80 | −73 | −75.8 | −78 |

### Example 7. Basic Bacteriological Characteristics of the Bifidobacterium animalis Subsp. lactis i797

This example illustrates the basic bacteriological characteristics of the Bifidobacterium animalis subsp. lactis i797 in the example 1, which are shown in Table 2:

TABLE 2
Basic Characteristics of the Bifidobacterium Animalis Subsp. Lactis i797

| Test item | Result | Test item | Result |
|---|---|---|---|
| Gram staining | Positive | Cell morphology | Polymorphic rod shape |
| Oxidase | — | Catalase | — |

### Example 8. Sugar Fermentation Characteristics of the Bifidobacterium Animalis Subsp. Lactis i797

This example illustrates the sugar fermentation characteristics of the strain of Bifidobacterium animalis subsp. lactis i797 in the example 1. The experimental method for sugar fermentation characteristics is as follows: a single colony of the strain of Bifidobacterium animalis subsp. lactis i797 obtained with the separation and purification method in the example 3 is inoculated into a sterilized modified MRS liquid culture medium, and cultured at 37° C. for 24 hours, then the bacterial suspension is inoculated into a sugar fermentation tube, and cultured under anaerobic condition at 37° C. for 48 hours, and the color change is observed. The identification result of the sugar fermentation characteristics is shown in Table 3:

TABLE 3
Identification Result of Sugar Fermentation Characteristics of the Bifidobacterium Animalis
Subsp. Lactis i797

| Test item | Result | Test item | Result | Test item | Result |
|---|---|---|---|---|---|
| Glycerol | − | Mannitol | − | Melezitose | − |
| Erythritol | − | Sorbitol | − | Raffinose | + |
| D-arabinose | − | α-methyl-mannoside | − | Starch | − |
| L-arabinose | − | α-methyl-glucoside | − | Glycogen | − |
| D-ribose | + | N-acetyl-glucosamine | − | Xylitol | − |
| D-xylose | + | Amygdalin | + | Gentiobiose | − |
| L-xylose | − | Arbutin | − | D-turanose | − |
| Adonitol | − | Esculin | + | D-lyxose | − |
| β-methyl-D-xyloside | − | Salicin | + | D-tagatose | − |
| D-galactose | − | Cellobiose | + | D-fucose | − |
| D-glucose | + | Maltose | + | L-fucose | − |
| D-fructose | − | Lactose | + | D-arabitol | − |
| D-mannose | − | Melibiose | + | L-arabitol | − |
| L-sorbose | − | Sucrose | + | Gluconate | − |
| L-rhamnose | − | Trehalose | + | 2-keto-gluconate | − |
| Dulcitol | − | Inulin | − | Inositol | − |

Note:
″+″ means utilization through fermentation;
″−″ means no utilization with fermentation.

The modified MRS liquid culture medium used in this example has the same composition as the modified MRS liquid culture medium in the example 2.

### Example 9. Molecular Biological Identification of the Strain of Bifidobacterium animalis subsp. lactis i797

The strain of Bifidobacterium animalis subsp. lactis i797 obtained with the separation and purification method in the example 5 is subjected to molecular biological identification, and through DNA extraction, PCR amplification, 16SrRNA sequencing, and NCBI Online Blast, it is finally determined as a Bifidobacterium animalis subsp. lactis.

Its 16SrRNA sequencing result is as follows:

ACGGCTCCCCCACAAGGGTCGGGCCACCGGCTTCGGGTGCTACCCACTTT
CATGACTTGACGGGCGGTGTGTACAAGGCCCGGGAACGCATTCACCGCGG
CGTTGCTGATCCGCGATTACTAGCGACTCCGCCTTCACGCAGTCGAGTTG
CAGACTGCGATCCGAACTGAGACCGGTTTTCAGCGATCCGCCCCACGTCA
CCGTGTCGCACCGCGTTGTACCGGCCATTGTAGCATGCGTGAAGCCCTGG
ACGTAAGGGGCATGATGATCTGACGTCATCCCCACCTTCCTCCGAGTTGA
CCCCGGCGGTCCCACATGAGTTCCCGGCATCACCCGCTGGCAACATGCGG
CGAGGGTTGCGCTCGTTGCGGGACTTAACCCAACATCTCACGACACGAGC
TGACGACGACCATGCACCACCTGTGAACCGGCCCCGAAGGGAAACCGTGT
CTCCACGGCGATCCGGCACATGTCAAGCCCAGGTAAGGTTCTTCGCGTTG
CATCGAATTAATCCGCATGCTCCGCCGCTTGTGCGGGCCCCCGTCAATTT
CTTTGAGTTTTAGCCTTGCGGCCGTACTCCCCAGGCGGGATGCTTAACGC
GTTGGCTCCGACACGGGACCCGTGGAAAGGGCCCCACATCCAGCATCCAC
CGTTTACGGCGTGGACTACCAGGGTATCTAATCCTGTTCGCTCCCCACGC
TTTCGCTCCTCAGCGTCAGTGACGGCCCAGAGACCTGCCTTCGCCATTGG
TGTTCTTCCCGATATCTACACATTCCACCGTTACACCGGGAATTCCAGTC
TCCCCTACCGCACTCCAGCCCGCCCGTACCCGGCGCAGATCCACCGTTAG
GCGATGGACTTTCACACCGGACGCGACGAACCGCCTACGAGCCCTTTACG
CCCAATAAATCCGGATAACGCTCGCACCCTACGTATTACCGCGGCTGCTG
GCACGTAGTTAGCCGGTGCTTATTCGAACAATCCACTCAACACGGCCGAA
ACCGTGCCTTGCCCTTGAACAAAAGCGGTTTACAACCCGAAGGCCTCCAT
CCCGCACGCGGCGTCGCTGCATCAGGCTTGCGCCCATTGTGCAATATTCC
CCACTGCTGCCTCCCGTAGGAGTCTGGGCCGTATCTCAGTCCCAATGTGG
CCGGTCACCCTCTCAGGCCGGCTACCCGTCAACGCCTTGGTGGGCCATCA
CCCCGCCAACAAGCTGATAGGACGCGACCCCATCCCATGCCGCAAAAGCA
TTTCCCACCCCACCATGCGATGGAGCGGAGCATCCGGTATTACGACCCGT
TTCCAGGAGCTATTCCGGTGCACAGGGCAGGTTGGTCACGCATTACTCAC
CCGTTCGCCACTCTCACCCGACAGCAAGCTGCCAGGGATCCCGTTCGACT
GCATGTGTAAG.

Its tuf gene sequencing result is as follows:

GGATCTCGATGAGAGCAGCGTGGTATCACCATCAACATTGCCCACATCGA
GTACCAGACGGCCAAGCGTCACTACGCCCACGTCGACTGCCCGGGCCACG
CCGACTTCGTGAAGAACATGATCACCGGCGCTGCCCAGATGGATGGCGCC
ATCCTCGTTGTGGCCGCCACCGACGGCCCGATGGCCCAGACCCGCGAGCA
CGTGCTGCTCGCCCGTCAGGTCGGCGTCCCGAAGATCCTCGTCGCTCTGA
ACAAGTGCGATATGGTCGATGACGAAGAGCTCATCGAGCTCGTCGAAGAA
GAGGTCCGCGACCTCCTCGACGAGAACGGCTTCGACCGCGACTGCCCGGT
CGTGCACACCTCCGCTTACGGCGCTCTGCATGACGACGCTCCCGGATCAC
GACAAGTGGGTTGCCACCATCAAGGAGCTCATGGACGACGTCGACGAGTA
CATCCCGACCCCGGTCCACGACCTCGACAAGCCGTTCCTGATGCCGATCG
AGGACGTCTTCACCATCTCCGGCCGTGGCACCGTCGTCACCGGTCGTGTC
GAGCGCGGCAAGCTGCCGATCAACACGAACGTCGAGATCGTCGGCATCCG
CCCGACCCAGACCACCACCGTCACCTCCATCGAGACCTTCCACAAGCAGA
TGGATGAGTGCGAGGCCGGCGACAACACCGGTCTGCTGCTCCGCGGCATC
AACCGCACCGACGTCGAGCGTGGCCAGGTCGTGGCTGCTCCGGGTTCGGT
CACCCCGCACACCAAGTTCGAAGGCGAAGTCTACGTCCTTACCAAGGATG
AGGGCGGCCGTCACTCGCCGTTCTTCTCGAACTACCGTCCGCAGTTCTAC
TTCCGCACCACCGACGTCACCGGCGTCATCACGCTGCCGGAAGGCGTCGA
GATGGTTCAGCCTGGCGATCACGCGACCTTCACGGTTGAGCTGATCCAGC
CGATCGCTATGGAAGAGGGCTTCACCTTCCCAGTGCTTGAAGGC.

### Example 10. Tolerance of the Bifidobacterium animalis Subsp. Lactis i797 to Gastric Fluid and Intestinal Fluid

After the strain of Bifidobacterium animalis subsp. lactis i797 to be tested is activated for 3 generations, 1 mL strain is injected in 9 mL filtered and sterilized artificial gastric fluid with a pH of 3.0, the mixture is shaken homogeneously and cultured in an anaerobic environment at 37° C.; samples are taken at the beginning of culturing and after 2 hours culturing respectively, and the viable counts are determined respectively. Then, 1 mL culture solution which has been digested in the artificial gastric fluid with 3.0 pH for 2 hours is inoculated into 9 mL filtered and sterilized artificial intestinal fluid with a pH of 8.0, and is further cultured at 37° C.; the viable count is measured at 0 hour, 4 hours and 6 hours respectively.

Bifidobacterium BB-12 is used as a standard strain for a control experiment, and the experimental parameters are the same as those of the experiment for the strain of Bifidobacterium animalis subsp. lactis i797.

Survival rate (%)=(cfu N₁/cfu N₀)×100%

where, N₁—the viable count after treatment with the artificial digestive fluid for 6 hours; N₀—the viable count after treatment with the artificial digestive fluid for 0 hour.
b. Intestinal fluid: 0.9 g Bile Salts (Difco) per 100 mL, the pH is adjusted to 8.0, and the fluid is filtered and sterilized for later use.

TABLE 4
Result of Tolerance of Bifidobacterium Animalis Lactis
Subsp. i797 to Simulated Gastric Fluid and Intestinal Fluid

| | | | | Survival | Survival | Survival |
|---|---|---|---|---|---|---|
| | | | | rate in | rate in | rate in |
| | | | | gastric | intestinal | digestive |
| Strain | 0 h | 2 h | 6 h | fluid | fluid | fluid |
| i797 | 5.0 × 10⁸ | 3.2 × 10⁶ | 3.7 × 10⁵ | 6.4% | 116% | 7.4% |
| BB-12 | 2.6 × 10⁸ | 1.8 × 10⁶ | 1.2 × 10⁵ | 6.8% | 66.7% | 4.5% |

The experiment demonstrates that BB-12 has strong tolerance to gastric acid and poor tolerance to intestinal fluid, while the Bifidobacterium animalis subsp. lactis i797 has poor tolerance to gastric acid and strong tolerance to intestinal fluid. By comprehensive comparison, the Bifidobacterium animalis subsp. lactis i797 achieves a better survival rate in the simulated digestive fluid, which is 7.4% and superior to that of BB-12.

### Example 11. Study on the Intestinal Regulation of the Bifidobacterium animalis Subsp. lactis i797

A drink containing the Bifidobacterium animalis subsp. lactis i797 is given to the testers for drinking, and statistics on the drinking result of 10 testers is carried out.

The statistical result is shown in Table 5:

TABLE 5
Analysis of Difference of Indexes after Drinking the Drink Containing
Bifidobacterium Animalis Subsp. Lactis i797

| | Drinking for a week | | Drinking for two weeks | |
|---|---|---|---|---|
| | | Is there a | | Is there a |
| Item | P value | difference | P value | difference |
| Frequency of | 0.024 | Yes | 0.009 | Yes |
| defecation | | | | |
| Difficulty in | 0.087 | No | 0.042 | Yes |
| defecation | | | | |
| Feeling after | 0.250 | No | 0.179 | No |
| defecation | | | | |
| Hardness of feces | 0.023 | Yes | 0.091 | No |
| Amount of feces | 0.655 | No | 0.281 | No |
| Odor of feces | 0.909 | No | 0.159 | No |
| Color of feces | 0.336 | No | 0.095 | No |
| Shape of feces | 0.016 | Yes | 0.107 | No |
| Gastrointestinal | 0.923 | No | 0.637 | No |
| condition | | | | |

Note:
Any significant change (statistically significant, P < 0.05) means there is a difference.

In the statistical process:
(1) Through analysis on the differences, it is found that there are significant differences in the frequency of defecation, difficulty in defecation, hardness of feces, and shape of feces;
(2) Through conversation with the drinkers, it is found that after drinking, the frequency of defecation is increased, the color of the feces turns yellow, and the feces became softer, indicating obvious change of indexes.

It can be seen from Table 5: among all indexes, four indexes (frequency of defecation, difficulty in defecation, hardness of feces, and shape of feces) have changed significantly (statistically significant, P<0.05), which indicates that Bifidobacterium animalis subsp. lactis i797 has a regulating effect on the intestinal tracts.

### Example 12. Analysis on Post-Acidification of the Strain of Bifidobacterium animalis subsp. lactis i797

Fresh milk of 97-98 parts and white sugar of 2-3 parts are mixed and blended evenly, homogenized at 65° C. and 15 MPa, sterilized at 95° C. for 300 seconds, and then cooled down to 37° C. to obtain sterilized milk; the Bifidobacterium animalis subsp. lactis i797 is inoculated into the sterilized milk with an inoculation amount of 3 wt % of the sterilized milk, and then fermented at 36° C., and the pH change is detected.

Standard strain of Bifidobacterium BB-12 is used as a control strain for control experiment, and the experimental parameters are the same as those of the above experiment for the strain of Bifidobacterium animalis subsp. lactis i797.

The result of acid production is shown in Table 6.

TABLE 6
Change of the Acidity of the Bifidobacterium Animalis Subsp. Lactis i797

| Fermentation time | BB-12 | JMCC0025 |
|---|---|---|
| 0 h | 6.4 | 6.32 |
| 18 h | 6.13 | 6.06 |
| 24 h | 5.55 | 4.66 |
| 42 h | 3.97 | 3.92 |
| 49 h | 3.86 | 3.87 |
| 66 h | 3.82 | 3.87 |
| 90 h | 3.82 | 3.88 |

It can be seen from Table 6: the Bifidobacterium animalis subsp. lactis i797 can control the post-acidification well under a storage condition suitable for the growth of lactic acid bacteria at a higher temperature of 37° C.

### Example 13. Use of the Strain of Bifidobacterium animalis Subsp. Lactis i797

This example provides use of the Bifidobacterium animalis subsp. lactis i797 in Example 1, which can be used to prepare drinks, foods or medicines. For example, the strain can be used to prepare cereals and their derivatives, fermented meat products, probiotics and formula milk powder, which have an intestinal regulation function; it can also be used to prepare beverages and fermented yogurts that have an intestinal regulation function; in addition, it can be used to prepare medicines with an intestinal regulation function, in a dosage form of capsule, powder, pill, oral liquid or spray, etc.

### Example 14. Use of the Strain of Bifidobacterium animalis Subsp. Lactis i797

The probiotics in the example 13 may be probiotics that only contain the Bifidobacterium animalis subsp. lactis i797; or the probiotics may be compound probiotics prepared by mixing Bifidobacterium animalis subsp. lactis i797, Lactobacillus paracasei N1115, Lactobacillus plantarum N3117 and Streptococcus thermophilus JMCC0003, and the dosage of the compound probiotics may be compound probiotic microcapsule powder.

1. A Bifidobacterium animalis subsp. lactis i797, characterized in that, the strain of Bifidobacterium animalis subsp. lactis i797 is preserved in the China General Microbiological Culture Collection Center, the preservation address is: No. 3, Yard No. 1, Beichen West Road, Chaoyang District, Beijing, the preservation date is Aug. 20, 2019, and the preservation number is CGMCC No. 18403.

2. The Bifidobacterium animalis subsp. lactis i797 according to claim 1, characterized in that, it is screened out from the intestinal flora of infants or babies.

3. The Bifidobacterium animalis subsp. lactis i797 according to claim 1, characterized in that, its 16SrRNA sequence is as follows:
ACGGCTCCCCCACAAGGGTCGGGCCACCGGCTTCGGGTGCTACCCACTTT
CATGACTTGACGGGCGGTGTGTACAAGGCCCGGGAACGCATTCACCGCGG
CGTTGCTGATCCGCGATTACTAGCGACTCCGCCTTCACGCAGTCGAGTTG
CAGACTGCGATCCGAACTGAGACCGGTTTTCAGCGATCCGCCCCACGTCA
CCGTGTCGCACCGCGTTGTACCGGCCATTGTAGCATGCGTGAAGCCCTGG
ACGTAAGGGGCATGATGATCTGACGTCATCCCCACCTTCCTCCGAGTTGA
CCCCGGCGGTCCCACATGAGTTCCCGGCATCACCCGCTGGCAACATGCGG
CGAGGGTTGCGCTCGTTGCGGGACTTAACCCAACATCTCACGACACGAGC
TGACGACGACCATGCACCACCTGTGAACCGGCCCCGAAGGGAAACCGTGT
CTCCACGGCGATCCGGCACATGTCAAGCCCAGGTAAGGTTCTTCGCGTTG
CATCGAATTAATCCGCATGCTCCGCCGCTTGTGCGGGCCCCCGTCAATTT
CTTTGAGTTTTAGCCTTGCGGCCGTACTCCCCAGGCGGGATGCTTAACGC
GTTGGCTCCGACACGGGACCCGTGGAAAGGGCCCCACATCCAGCATCCAC
CGTTTACGGCGTGGACTACCAGGGTATCTAATCCTGTTCGCTCCCCACGC
TTTCGCTCCTCAGCGTCAGTGACGGCCCAGAGACCTGCCTTCGCCATTGG
TGTTCTTCCCGATATCTACACATTCCACCGTTACACCGGGAATTCCAGTC
TCCCCTACCGCACTCCAGCCCGCCCGTACCCGGCGCAGATCCACCGTTAG
GCGATGGACTTTCACACCGGACGCGACGAACCGCCTACGAGCCCTTTACG
CCCAATAAATCCGGATAACGCTCGCACCCTACGTATTACCGCGGCTGCTG
GCACGTAGTTAGCCGGTGCTTATTCGAACAATCCACTCAACACGGCCGAA
ACCGTGCCTTGCCCTTGAACAAAAGCGGTTTACAACCCGAAGGCCTCCAT
CCCGCACGCGGCGTCGCTGCATCAGGCTTGCGCCCATTGTGCAATATTCC
CCACTGCTGCCTCCCGTAGGAGTCTGGGCCGTATCTCAGTCCCAATGTGG
CCGGTCACCCTCTCAGGCCGGCTACCCGTCAACGCCTTGGTGGGCCATCA
CCCCGCCAACAAGCTGATAGGACGCGACCCCATCCCATGCCGCAAAAGCA
TTTCCCACCCCACCATGCGATGGAGCGGAGCATCCGGTATTACCACCCGT
TTCCAGGAGCTATTCCGGTGCACAGGGCAGGTTGGTCACGCATTACTCAC
CCGTTCGCCACTCTCACCCGACAGCAAGCTGCCAGGGATCCCGTTCGACT
GCATGTGTAAG.

4. The Bifidobacterium animalis subsp. lactis i797 according to claim 1, characterized in that, its tuf gene sequence is as follows:
GGATCTCGATGAGAGCAGCGTGGTATCACCATCAACATTGCCCACATCGA
GTACCAGACGGCCAAGCGTCACTACGCCCACGTCGACTGCCCGGGCCACG
CCGACTTCGTGAAGAACATGATCACCGGCGCTGCCCAGATGGATGGCGCC
ATCCTCGTTGTGGCCGCCACCGACGGCCCGATGGCCCAGACCCGCGAGCA
CGTGCTGCTCGCCCGTCAGGTCGGCGTCCCGAAGATCCTCGTCGCTCTGA
ACAAGTGCGATATGGTCGATGACGAAGAGCTCATCGAGCTCGTCGAAGAA
GAGGTCCGCGACCTCCTCGACGAGAACGGCTTCGACCGCGACTGCCCGGT
CGTGCACACCTCCGCTTACGGCGCTCTGCATGACGACGCTCCCGGATCAC
GACAAGTGGGTTGCCACCATCAAGGAGCTCATGGACGACGTCGACGAGTA
CATCCCGACCCCGGTCCACGACCTCGACAAGCCGTTCCTGATGCCGATCG
AGGACGTCTTCACCATCTCCGGCCGTGGCACCGTCGTCACCGGTCGTGTC
GAGCGCGGCAAGCTGCCGATCAACACGAACGTCGAGATCGTCGGCATCCG
CCCGACCCAGACCACCACCGTCACCTCCATCGAGACCTTCCACAAGCAGA
TGGATGAGTGCGAGGCCGGCGACAACACCGGTCTGCTGCTCCGCGGCATC
AACCGCACCGACGTCGAGCGTGGCCAGGTCGTGGCTGCTCCGGGTTCGGT
CACCCCGCACACCAAGTTCGAAGGCGAAGTCTACGTCCTTACCAAGGATG
AGGGCGGCCGTCACTCGCCGTTCTTCTCGAACTACCGTCCGCAGTTCTAC
TTCCGCACCACCGACGTCACCGGCGTCATCACGCTGCCGGAAGGCGTCGA
GATGGTTCAGCCTGGCGATCACGCGACCTTCACGGTTGAGCTGATCCAGC
CGATCGCTATGGAAGAGGGCTTCACCTTCCCAGTGCTTGAAGGC.

5. A method for separating and purifying the Bifidobacterium animalis subsp. lactis i797 according to claim 1, characterized in that, it comprises the following steps which are carried out in sequence:
I. sample collection
obtaining intestinal feces of infants or babies, then adding the intestinal feces into normal saline and mix thoroughly to obtain a sample A;
II. sample enrichment
adding the sample A into a modified MRS liquid culture medium, and culturing in an anaerobic environment at 35-40° C. for 62-82 hours to obtain a culture solution B;
the modified MRS liquid culture medium is an MRS liquid culture medium added with 0.5 wt % cysteine;
the volume ratio of the sample A to the modified MRS liquid culture medium is 1:10-100;
III. strain separation and screening
diluting the culture solution B with 0.9% sterile normal saline by ten-time gradient multiplication, i.e., diluting to 10⁻¹, 10⁻², 10⁻³, 10⁻⁴ and 10⁻⁵ times sequentially, thus obtaining bacterial suspensions C₁-C₅ correspondingly;
melting modified MRS solid culture medium and pouring it into the first to fifth culture dishes, thus obtaining culture media D₁-D₅ after cooling and complete solidification; drawing 0.1 mL bacterial suspensions C₁-C₅ and spreading them on the culture media D₁-D₅ respectively in one-to-one correspondence, then turning the plates upside down and culturing them in an anaerobic environment at 35-40° C. for 62-82 hours, and observing the growth of the colonies;
the modified MRS solid culture medium is a solid culture medium obtained by adding 15 wt % agar per 1,000 mL modified MRS liquid culture medium;
after typical colonies appear on the plates, picking corresponding single colony E;
IV. strain purification
picking a selected single colony E and streak-inoculating the single colony E culture on the modified MRS solid culture medium, and culturing in anaerobic environment at 35-40° C. for 62-82 hours to obtain a single colony F;
streak-inoculating the single colony F further on the modified MRS solid culture medium, and culturing in an anaerobic environment at 35-40° C. for 62-82 hours to obtain a single colony G;
streak-inoculating the single colony G further on the modified MRS solid culture medium, and culturing in an anaerobic environment at 35-40° C. for 62-82 hours to obtain a pure culture H, which is the strain of Bifidobacterium animalis subsp. lactis i797.

6. The method for separating and purifying the Bifidobacterium animalis subsp. lactis i797 according to claim 5, wherein the strain of Bifidobacterium animalis subsp. lactis i797 is preserved as follows: mixing the pure culture H with 50 wt % sterile glycerol at a ratio of 1:1, placing the mixture in a strain preservation tube, mixing homogeneously and then preserving at −80-70° C.; at the same time, inoculating on test-tube slant of the modified MRS solid culture medium for temporary storage.

7. The method for separating and purifying the Bifidobacterium animalis subsp. lactis i797 according to claim 4, wherein the components of the modified MRS liquid culture medium include: casein peptone, beef extract, yeast extract, glucose, sodium acetate, diamine citrate, Tween-80, K₂HPO₄, MgSO₄.7H₂O, MnSO₄.7H₂O, cysteine and distilled water;
wherein the dosage ratio of casein peptone:beef extract:yeast extract:glucose:sodium acetate:diamine citrate:Tween-80:K₂HPO₄:MgSO₄.7H₂O:MnSO₄.7H₂O:cysteine:distilled water is 10 g:10 g:5 g:20 g:5 g:2 g:1 g:2 g:0.2 g:0.05 g: 0.5 g:1,000 mL.

8. Use of the Bifidobacterium animalis subsp. lactis i797 according to claim 1 in preparation of drinks, foods or medicines.

9. The use of the Bifidobacterium animalis subsp. lactis i797 according to claim 8,
wherein the drinks are beverages or fermented milk drinks;
the foods are cereals, cereal derivatives, fermented meat products, probiotics or milk foods;
the medicines are in dosage forms of capsule, tablet, pill or powder.

10. The use of the Bifidobacterium animalis subsp. lactis i797 according to claim 9, wherein the probiotics are compound probiotics.
